(19) United States
(12) Patent Application Publication
(10) Pub. No.: US 2025/0389647 A1
(43) Pub. Date: Dec. 25, 2025

(54) PROCESS EVALUATION APPARATUS, PROCESS EVALUATION METHOD, AND PROCESS EVALUATION PROGRAM

(71) Applicant: HORIBA STEC, Co., Ltd., Kyoto (JP)

(72) Inventors: Toru SHIMIZU, Kyoto (JP); Miyako HADA, Kyoto (JP)

(73) Assignee: HORIBA STEC, Co., Ltd., Kyoto (JP)

(21) Appl. No.: 19/230,698

(22) Filed: Jun. 6, 2025

(30) Foreign Application Priority Data
Jun. 21, 2024 (JP) 2024-100475

(51) Int. Cl. G01N 21/31; G01N 33/00; H01J 37/305

(52) U.S. Cl. CPC G01N 21/31; G01N 33/004; G01N 2201/06113; H01J 37/3053

(57) ABSTRACT

The present invention makes it possible for the fabrication time or fabrication conditions or the like in at least one of a deposition step, an anisotropic etching step, or an isotropic etching step to be appropriately adjusted, and is provided with light absorption measurement units 2A and 2B that irradiate light onto a gas containing a reaction product that has been generated during a process, and then measure the reaction product based on an absorption of this light, and with a state evaluation unit 3 that, based on measurement values obtained by the light absorption measurement units 2A and 2B, evaluates a state of the process.

### CROSS-REFERENCE TO RELATED APPLICATION

The present application claims priority of Japanese Application No. 2024-100475, filed on Jun. 21, 2024, the entire contents of which is hereby incorporated by reference in its entirety.

### BACKGROUND OF THE INVENTION

### 1. Technical Field

The present invention relates to a process evaluation apparatus, a process evaluation method, and a process evaluation program.

### 2. Description of the Related Art

Conventionally, deep reactive-ion etching (deep RIE) is used in order to perform microfabrication on a silicon substrate. Note that deep RIE is reactive-ion etching (RIE) having a high aspect ratio (i.e., narrow and deep).

As is shown in FIG. 11, among this deep RIE is a process (known as a Bosch process) that includes repeatedly performing a deposition step in which a protective film is deposited on a substrate, an anisotropic etching step in which a bottom surface of the protective film is removed by means of anisotropic etching, and an isotropic etching step in which the substrate that has been exposed due to the protective film being removed is removed by means of isotropic etching (see, for example, Patent Document 1). If this Bosch process is employed, then it is possible to form a deep groove or deep hole by cutting away the bottom surface of the groove or hole while protecting side surfaces of the groove or hole by means of the protective film.

However, if the fabrication time or fabrication conditions or the like in at least one of the aforementioned deposition step, anisotropic etching step, or isotropic etching step are not appropriately adjusted, then it becomes no longer possible to hollow out the grooves or holes in parallel. If this occurs, the grooves or holes end up having a tapered shape or an inverse tapered shape or, alternatively, effects such as mutually adjacent grooves or holes becoming joined together or the like are generated.

### PRIOR ART DOCUMENT

### Patent Document

Patent Document 1: Japanese Patent Application (JP-A) Laid-Open No. 2019-102593

### SUMMARY OF THE INVENTION

The present invention was, therefore, conceived in order to solve the above-described problem, and it is a principal object thereof to make it possible for the fabrication time or fabrication conditions or the like in at least one of a deposition step, an anisotropic etching step, or an isotropic etching step in a process to be appropriately adjusted.

In other words, a process evaluation apparatus according to the present invention is a process evaluation apparatus that evaluates a process in which are repeatedly performed a deposition step in which a protective film is deposited on a substrate, an anisotropic etching step in which a portion of the protective film is removed by means of anisotropic etching, and an isotropic etching step in which the substrate that has been exposed due to the protective film being removed is removed by means of isotropic etching, and that is characterized in being provided with light absorption measurement units that irradiate light onto a gas containing a reaction product that has been generated during the process, and then measure the reaction product based on an absorption of this light, and a state evaluation unit that, based on measurement values obtained by the light absorption measurement units, evaluates a state of the process.

According to this process evaluation apparatus, because a reaction product is measured by irradiating light onto a gas containing a reaction product that has been generated during a process, and a state of the process is then evaluated based on measurement values of the reaction product, it is possible for the fabrication time or fabrication conditions or the like in at least one of a deposition step, an anisotropic etching step, or an isotropic etching step in what is known as a Bosch process to be appropriately adjusted. As a result, it is possible to make the fabricated profile of a groove or hole being formed in a substrate in a Bosch process a desired profile, and to thereby improve the performance of a device that utilizes the substrate thus fabricated.

The aforementioned process is employed in order to etch silicon. In this case, using C₄F₈ as the processing gas in the protective film deposition step, and using SF₆ as the processing gas in the anisotropic etching and in the isotropic etching may be considered. In this case, it is desirable that the light absorption measurement units measure a first reaction product (for example, SiF₄) generated as a result of the silicon being etched, and that the state evaluation unit evaluate the state of the process based on measurement values of the first reaction product obtained by the light absorption measurement units.

As a result of using light absorption measurement units, the inventors of the present application discovered that the first reaction product (for example, SiF₄) was generated not only in the anisotropic etching and isotropic etching, but was also generated in the deposition step. They considered that the reason for this was that the Si exposed by plasma during the deposition step had reacted and thereby caused the first reaction product (for example, SiF₄) to be generated.

Because of this, the state evaluation unit evaluates the state of the process based on measurement values of the first reaction product (for example, SiF₄) obtained during the deposition step.

As a specific aspect of the state evaluation unit, it is desirable that the state evaluation unit detect an end point of the process based on a size of a rise in the measurement values of the first reaction product (for example, SiF₄) obtained during the deposition step.

As a specific aspect of the state evaluation unit, in a case in which the measurement values of the first reaction product (for example, SiF₄) obtained during the deposition step are equal to or less than a threshold value, it is desirable that the state evaluation unit determine that a deposition of the protective film is sufficient.

Conventionally, because the first reaction product (for example, SiF₄) is generated during the anisotropic etching step or the isotropic etching step, it is desirable that the state evaluation unit evaluate the state of the process based on measurement values of the first reaction product (for example, SiF₄) obtained during the anisotropic etching step or the isotropic etching step.

As a specific aspect of the state evaluation unit, it is desirable that the state evaluation unit detect that an object being etched has switched from the protective film to silicon based on measurement values of the first reaction product (for example, SiF₄) obtained during the anisotropic etching step.

As a specific aspect of the state evaluation unit, it is desirable that the state evaluation unit detect an end point of the process based on measurement values of the first reaction product (for example, SiF₄) obtained during the anisotropic etching step or the isotropic etching step.

Moreover, it is also desirable that the protective film be formed by a compound that contains carbon, and that the light absorption measurement units measure a second reaction product (for example, CO, CO₂, or CF₄) that is generated as a result of the protective film being etched, and that, in a case in which measurement values of the second reaction product obtained by the light absorption measurement units during the isotropic etching step are equal to or less than a threshold value, the state evaluation unit determine that the protective film is insufficient.

It is also desirable that the light absorption measurement units be provided in an exhaust pipe that is connected to a chamber where the process is performed.

If this type of structure is employed, then compared with a reaction product inside a chamber, a reaction product flowing through an exhaust pipe is in a more stable state, and it is possible to evaluate the state of a process more accurately by measuring the reaction product in the exhaust pipe. Note that an apparatus that detects light emissions may be considered as a process monitor, however, because no light emissions are generated from a stable reaction product they cannot be measured.

It is also desirable that the light absorption measurement units irradiate laser light onto the gas, and measure the reaction product based on an absorption of that laser light.

If this structure is employed, then because laser light moves in a straight direction, even if a long light-path cell is used, it is still easy to achieve a high degree of sensitivity and to thereby evaluate a process more appropriately.

Moreover, a process evaluation method according to the present invention is a process evaluation method in which is evaluated a process in which are repeatedly performed a deposition step in which a protective film is deposited on a substrate, an anisotropic etching step in which a portion of the protective film is removed by means of anisotropic etching, and an isotropic etching step in which the substrate that has been exposed due to the protective film being removed is removed by means of isotropic etching, and which is characterized in that light is irradiated onto a gas containing a reaction product that has been generated during the process, and the reaction product is then measured based on an absorption of this light, and a state of the process is evaluated based on measurement values of the reaction product.

Furthermore, a process evaluation program of the present invention is a process evaluation program for evaluating a process in which are repeatedly performed a deposition step in which a protective film is deposited on a substrate, an anisotropic etching step in which a portion of the protective film is removed by means of anisotropic etching, and an isotropic etching step in which the substrate that has been exposed due to the protective film being removed is removed by means of isotropic etching, and that is characterized by enabling a computer to function as a state evaluation unit that evaluates a state of the process based on measurement values obtained by light absorption measurement units that irradiate light onto a gas containing a reaction product that has been generated during the process, and then measure the reaction product.

According to the present invention which is formed in the manner described above, it is possible for the fabrication time or fabrication conditions or the like in at least one of a deposition step, an anisotropic etching step, or an isotropic etching step in a process to be appropriately adjusted.

### BRIEF DESCRIPTION OF THE DRAWINGS

FIG. 1 is a schematic diagram showing a process evaluation apparatus according to an embodiment of the present invention;

FIG. 2 is a functional structure diagram of a signal processing device and a calculation device of light absorption measurement units in the same embodiment;

FIG. 3 is a schematic diagram showing a method of modulating a laser oscillation wavelength in the same embodiment;

FIG. 4 is a time series graph showing an example of an oscillation wavelength, a light intensity I (t), a logarithmic intensity L (t), a feature signal Fᵢ (t), and a correlation value Sᵢ in the same embodiment;

FIG. 5 shows conceptual views of a concentration or partial pressure calculation utilizing individual correlation values and sample correlation values in the same embodiment;

FIG. 6 shows (a) schematic views before and after a deposition step, and (b) graphs of measurement values of SiF₄ in the same embodiment;

FIG. 7 shows graphs of (a) measurement values of SiF₄ in a case in which a protective film is insufficient, and (b) measurement values of SiF₄ in a case in which a protective film is sufficient in the same embodiment;

FIG. 8 shows (a) schematic views before and after an anisotropic etching step, and (b) graphs of measurement values of SiF₄ in the same embodiment;

FIG. 9 shows graphs of (a) measurement values of CO in a case in which a protective film is insufficient, and (b) measurement values of CO in a case in which a protective film is sufficient in the same embodiment;

FIG. 10 shows (a) a graph of measurement values of SiF₄ and CO in a case in which a protective film is insufficient as well as an etching profile at that time, and (b) a graph of measurement values of SiF₄ and CO in a case in which a protective film is sufficient as well as an etching profile at that time; and

FIG. 11 is a schematic view showing each step of a Bosch process.

### DETAILED DESCRIPTION

Hereinafter, an embodiment of a process evaluation apparatus according to the present invention will be described with reference to the drawings.

Note that, in order to simplify an understanding thereof, each of the drawings depicted below is shown schematically with omissions or enhancements made where these have been deemed appropriate. In addition, component elements that are the same in the respective drawings are indicated by the same descriptive symbols and any duplicated description thereof is omitted.

[Apparatus Structure]

A process evaluation apparatus of the present embodiment evaluates a process by measuring a reaction product generated during that process.

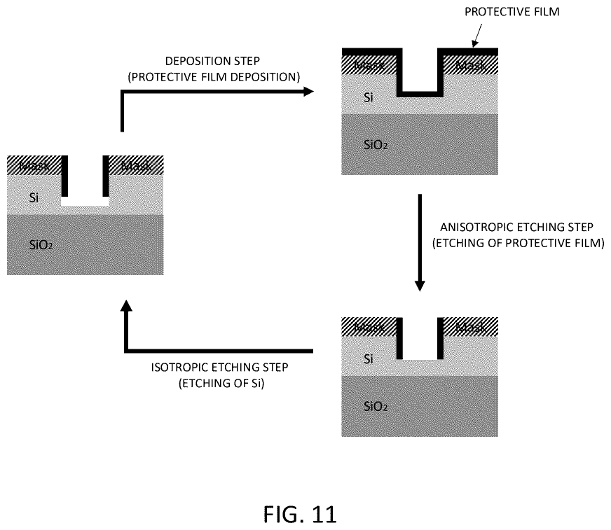

Here, the process that is being evaluated is what is known as a Bosch process and, as is shown in FIG. 11, is a process for forming a groove or hole having a high aspect ratio (i.e., that is narrow and deep) in a substrate in which are repeated a deposition step in which a protective film is deposited on a substrate, an anisotropic etching step in which a portion of the protective film is removed by anisotropic etching, and an isotropic etching step in which the substrate exposed as a result of the protective film being removed is then removed by isotropic etching. The substrate in the present embodiment has a SiO₂ layer, a silicon (Si) layer that is formed on an upper surface of this SiO₂ layer, and a mask layer that is formed in portions on an upper surface of this Si layer.

The deposition step is a step in which a CF-based polymer film that will serve as a protective film is deposited on an upper surface of a substrate by supplying, for example, C₄F₈ and O₂ as processing gases to the inside of a chamber where plasma has been created. Note that there are also cases in which O₂ is not used as a processing gas in the deposition step, and cases in which CF₄ is used instead of C₄F₈. In contrast, the anisotropic etching step is a step in which a portion of the protective film (namely, the protective film located in a bottom surface of a groove or hole) is removed by performing etching using F ions by supplying, for example, SF₆ and O₂ as processing gases to the inside of a chamber where plasma has been created and then applying a bias voltage to the substrate. Note that there are also cases in which O₂ is not used as a processing gas in the anisotropic etching step. Moreover, the isotropic etching step is a step in which the Si layer of the substrate (i.e., the bottom surface of a groove or hole) that has been exposed as a result of the protective film being removed is removed by performing etching using F radicals by supplying, for example, SF₆ and O₂ as processing gases to the inside of a chamber where plasma has been created. Note that there are also cases in which O₂ is not used as a processing gas in the isotropic etching step.

In the anisotropic etching step and isotropic etching step, the F ions and/or F radicals generated from the SF₆ that is serving as a processing gas react with the Si so that SiFₓ (for example, SiF, SiF₂, SiF₃, and SiF₄ and the like) and the like are produced as by-products. Because it is not possible for SiF, SiF₂, and SiF₃ to be present with any stability, in a case in which a processing state is being evaluated, it is considered desirable to measure SiF₄. Moreover, in the anisotropic etching step and isotropic etching step, the F ions and/or F radicals generated from the SF₆ that is serving as a processing gas react with the CF-based polymer film serving as a protective film so that CₓF_{y} (for example, CF, CF₂, CF₃, and CF₄ and the like) and the like are generated. In a case in which O₂ is used as a processing gas, COₓF_{y} (for example, CO, CO₂, COF, and COF₂ and the like) and the like may be generated. Of these reaction products, provided that the substance is present as a gas and has a measurable absorption (namely, CF₄, CO, CO₂, or COF₂ out of those mentioned above), then it is possible for the state of the processing to be evaluated.

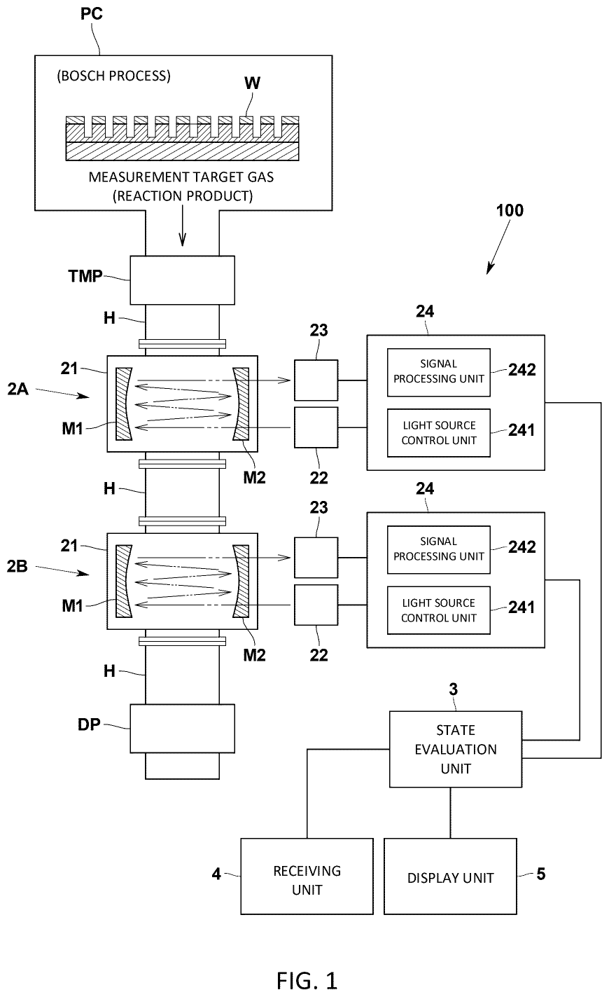

More specifically, as is shown in FIG. 1, a process evaluation apparatus 100 is provided with two light absorption measurement units 2A and 2B that measure a reaction product generated during a Bosch process, and with a state evaluation unit 3 that, based on measurement values obtained by the two light absorption measurement units 2A and 2B, evaluates a state of the Bosch process.

The respective light absorption measurement units 2A and 2B measure a reaction product by irradiating laser light onto a gas that contains a reaction product generated in a Bosch process, and then measuring the resulting laser light absorption. The light absorption measurement unit 2A of the present embodiment (hereinafter, this may also be referred to as a first light absorption measurement unit 2A) measures SiF₄ which is a first reaction product that is generated as a result of silicon being etched, while the laser absorption measurement unit 2B (hereinafter, this may also be referred to as a second light absorption measurement unit 2B) measures CO which is a second reaction product that is generated as a result of the protective film being etched.

As is shown in FIG. 1, the light absorption measurement units 2A and 2B are incorporated into an exhaust pipe H of a chamber PC where Bosch processing is performed, and have a structure that enables them to analyze a reaction product contained in a gas flowing through the exhaust pipe H (hereinafter, referred to as a measurement target gas). Note that, in the present embodiment, a turbo molecular pump TMP and a dry pump DP are provided on the exhaust pipe H, and the light absorption measurement units 2A and 2B are disposed between the turbo molecular pump TMP and the dry pump DP, however, the present invention is not limited to this.

### [Structure of the Light Absorption Measurement Units 2A and 2B]

The light absorption measurement units 2 continuously measure the concentration of reaction products (in this case, SiF₄ and CO) contained in the measurement target gas and employ, for example, infrared laser absorption modulation (IRLAM: see Japanese Patent No. 6886507) to achieve this.

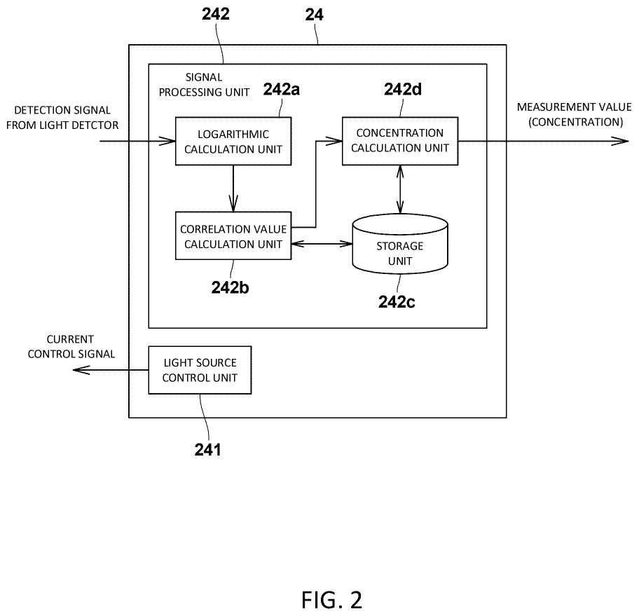

More specifically, as is shown in FIG. 1 and FIG. 2, the light absorption measurement units 2A and 2B are provided with measurement cells 21 that each have a pair of multiple reflection mirrors M1 and M2 that are disposed on either side of the measurement target gas, semiconductor lasers 22 that irradiate laser light into the measurement cells 21 so as to cause this laser light to be incident between the pair of multiple reflection mirrors M1 and M2, light detectors 23 that detect laser light that has exited from between the pair of multiple reflection mirrors M1 and M2 and has passed through the measurement cells 21, and signal processing devices 24 that calculate a concentration or partial pressure of the reaction products based on detection signals from the light detectors 23.

The measurement cells 21 are what are known as Herriott cells that, as a result of having the pair of multiple reflection mirrors M1 and M2 provided internally therein, reflect the laser light multiple times. Note that, instead of Herriott cells, the measurement cells 21 may also be formed by White cells having a plurality of multiple reflection mirrors that are disposed on either side of the measurement target gas, or ring cells that have a circular multiple reflection mirror that surrounds the measurement target gas.

The semiconductor lasers 22 are quantum cascade lasers. A quantum cascade laser is a semiconductor laser that uses an intersubband transition based on a multi-stage quantum well structure, and oscillates laser light having a specific wavelength in a wavelength range of between approximately 4 μm and 20 μm. This semiconductor laser 22 is able to modulate (i.e., alter) the oscillation wavelength by means of the supplied current (or voltage). Note that the semiconductor laser 22 of the first laser absorption measurement unit 2A oscillates laser light in the absorption wavelength band of SiF₄, while the semiconductor laser 22 of the second laser absorption measurement unit 2B oscillates laser light in the absorption wavelength band of CO.

The light detectors 23 used here are thermal light detectors such as comparatively low-cost thermopiles or the like, however, it is also possible for a different type of light detector such as, for example, a quantum photoelectric element such as HgCdTe, InGaAs, InAsSb, or PbSe or the like which are highly responsive to be used instead.

The signal processing devices 24 are equipped with an analog electrical circuit formed by amplifiers and the like, a CPU, digital electrical circuits formed by memory and the like, and AD converters and DA converters and the like that are interposed between these analog and digital electrical circuits.

As is shown in FIG. 2, as a result of the CPU and peripheral devices thereof operating in mutual collaboration with each other in accordance with a predetermined program that is stored in a predetermined area of the memory, each signal processing device 24 performs the functions of a light source control unit 241 that controls outputs from the semiconductor lasers 22, and of a signal processing unit 242 that receives detection signals from the light detectors 23 and performs arithmetic processing on the values contained therein so as to calculate concentrations or partial pressures of a measurement target component or values relating thereto. Note that these values relating to the concentrations or partial pressures include values having a correlation with the concentrations or partial pressures such as, for example, an absorption intensity or the like.

Each of these units will now be described in detail. In the example described below, the signal processing units 242 calculate a concentration of a measurement target component.

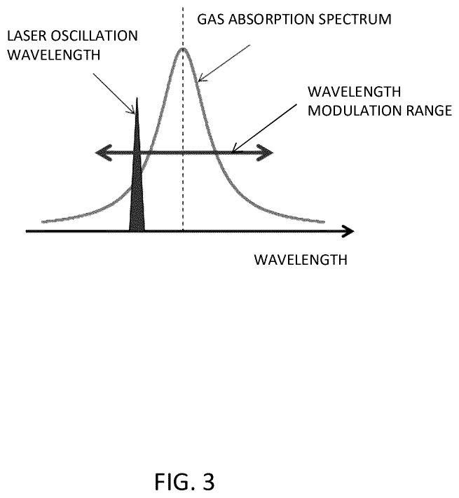

The light source control units 241 control a current source (or a voltage source) of each semiconductor laser 22 outputting current (or voltage) control signals. More specifically, each light source control unit 241 alters a drive current (or drive voltage) of the semiconductor laser 22 using a predetermined frequency so that the oscillation wavelength of the laser light output from the semiconductor laser 22 is modulated at a predetermined frequency relative to a central wavelength (see FIG. 3). As a result, each semiconductor laser 22 emits modulation light that has been modulated at a predetermined modulation frequency.

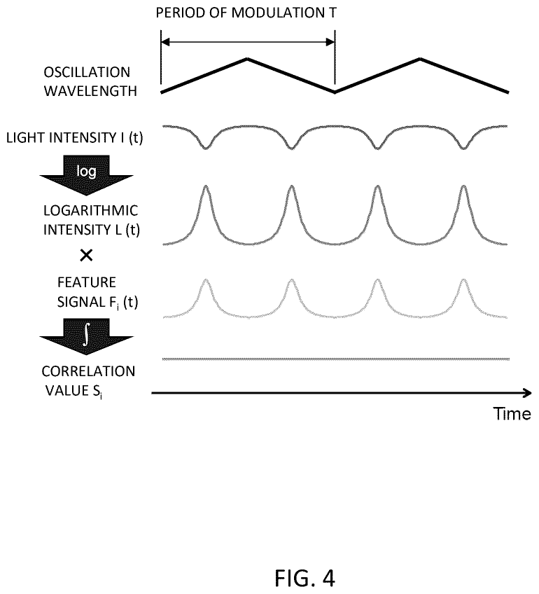

In this embodiment, each light source control unit 241 changes the drive current to a triangular waveform, and modulates the oscillation frequency to a triangular waveform (see ‘oscillation wavelength’ in FIG. 4). In actual fact, modulation of the drive current is performed using a separate function so that the oscillation wavelength attains a triangular waveform. Moreover, as is shown in FIG. 3, the oscillation wavelength of the laser light is modulated with a peak of a light absorption spectrum of the measurement target component taken as the central wavelength. In addition to this, it is also possible for each light source control unit 241 to change the drive current to a sinusoidal wave shape or a sawtooth wave shape, or to an arbitrary function shape, and to modulate the oscillation frequency to a sinusoidal wave shape or a sawtooth wave shape, or to an arbitrary function shape.

The signal processing units 242 are each formed by a logarithmic calculation unit 242a, a correlation value calculation unit 242b, a storage unit 242c, and a concentration calculation unit 242d and the like.

Each logarithmic calculation unit 242a performs logarithmic calculation processing on the light intensity signal that is formed by a detection signal from the light detector 23. A function I (t) that shows changes over time in a light intensity signal obtained by the light detector 23 has the form shown by ‘Light intensity I (t)’ in FIG. 4, and subsequently takes the form shown by ‘Logarithmic intensity L (t)’ in FIG. 4 as a result of a logarithmic calculation being performed.

Each correlation value calculation unit 242b calculates respective correlation values between intensity related signals which relate to the intensity of the sample light obtained at the time when the measurement target gas was being measured and a plurality of predetermined feature signals. These feature signals are signals that are used to extract a waveform feature of an intensity related signal by obtaining a correlation between the feature signal and the intensity related signal. Examples of feature signals include sinusoidal signals, and various signals that are matched to waveform features to be extracted from other intensity related signals. Here, a correlation value calculation unit 242b uses the light intensity signal ‘Logarithmic intensity L (t)’ that was obtained via the aforementioned logarithmic calculation as the intensity related signal.

In addition, each correlation value calculation unit 242b calculates a plurality of sample correlation values Sᵢ which are the respective correlation values between the intensity related signals of the sample light and the plurality of feature signals by employing the following (Equation 1) using a number of feature signals Fᵢ (t) (i=1, 2, . . . , n) that is greater than a number obtained by adding together the number of types of measurement target components (i.e., reaction products in the present embodiment) with the number of types of interference components. Note that, in Equation 1, T is the period of modulation.

$$\begin{matrix}{S}_{i} = {\int }_{0}^{T} L ( t ) \cdot {F}_{i} ( t ) dt & ( i = 1 , 2 , … , n )\end{matrix} \qquad [ Equation 1 ]$$

$${R}_{i} = {\int }_{0}^{T} {L}_{0} ( t ) \cdot {F}_{i} ( t ) dt \qquad ( i = 1 , 2 , … , n )$$

$${{S}_{i}}^{′} = {S}_{i} - {R}_{i}$$

It is desirable that, when calculating the sample correlation value, the correlation value calculation units 242b calculate a sample correlation value Sᵢ′ which, as is shown in Equation 1, is a corrected value obtained by subtracting a reference correlation value Rᵢ, which is a correlation value between an intensity related signal L₀ (t) of reference light and the plurality of feature signals Fᵢ (t), from the correlation value Sᵢ between the intensity related signal L (t) of the sample light and the plurality of feature signals Fᵢ (t). As a result, any offset contained in the sample correlation values is removed, and the correlation values are made proportional to the concentrations of the measurement target components and the interference components, thereby enabling measurement errors to be decreased. Note that it is also possible to employ a structure in which a reference correlation value is not subtracted.

Here, the acquisition timing of the reference light may be simultaneous with the acquisition of the sample light, or pre/post measurement, or another arbitrary timing. It is also possible for the intensity related signals of the reference light or the reference correlation values to be acquired in advance and stored in the storage units 242c. Moreover, a method that may be considered in order to acquire the reference light simultaneously with the sample light is a method in which two light detectors 23 are provided, and the modulation light from the semiconductor laser 22 is split by means of a beam splitter or the like, with one beam used for measuring the sample light, and the other beam used for measuring the reference light.

In the present embodiment, the correlation value calculation units 242b use, as the plurality of feature signals Fᵢ (t), a function from which a waveform characteristic of the logarithmic intensity L (t) is more easily obtained than from a sinusoidal function. In the case of a sample gas containing measurement target components and a single interference component, using two or more feature signals F₁ (t), F₂ (t) may be considered, and using, for example, a function based on a Lorentz function that is close to the shape of the absorption spectrum, and a differential function of this function that is based on the Lorentz function as the two feature signals F₁ (t), F₂ (t) may also be considered. Moreover, as the feature signals, instead of using a function that is based on a Lorentz function, it is also possible to use a function that is based on a Voigt function, or a function that is based on a Gaussian function or the like. By using a function of this type for the feature signal, it is possible to obtain a larger correlation value than when a sinusoidal function is used so that, as a consequence, the measurement accuracy can be improved.

Here, it is desirable that DC components be removed from the feature signals, in other words, that the offset be adjusted so that the offset is zero when integrated in the period of modulation. By employing this method, it is possible to remove any effects that might result when offset is superimposed on the intensity related signal due to variations in the light intensity. Note that, instead of removing DC components from the feature signals, it is also possible to remove DC components from the intensity related signals, or to remove DC components from both the feature signals and the intensity related signals. In addition, as the feature signals, it is also possible to use sample values of an absorption signal of the measurement target components and/or interference components, or, alternatively, to use values that are based on each of these.

Note also that by forming the two feature signals F₁ (t), F₂ (t) as an orthogonal function sequence in which the two feature signals F₁ (t), F₂ (t) are mutually orthogonal, or as a function sequence that is close to an orthogonal function sequence, it is possible to more efficiently extract the features of the logarithmic intensity L (t), and the concentrations obtained by means of simultaneous equations (described below) can be made more accurate.

The storage units 242c store individual correlation values which are correlation values per unit concentration between the measurement target components and the respective interference components that are determined from the respective intensity related signals and the plurality of feature signals Fᵢ (t) in a case in which the measurement target components and the respective interference components are present individually. The plurality of feature signals Fᵢ (t) that are used to determine this individual correlation value are the same as the plurality of feature signals Fᵢ (t) that are used in the correlation value calculation units 242b.

Here, it is desirable that, when storing the individual correlation values, the storage units 242c store individual correlation values that have been corrected by being converted into per unit concentrations after the reference correlation value has been subtracted from the correlation values in a case in which the measurement subject component and the respective interference components are present individually. As a result, any offset contained in the individual correlation values is removed, and the correlation values are made proportional to the concentrations of the measurement target components and the interference components, thereby enabling measurement errors to be decreased. Note that it is also possible to employ a structure in which the reference correlation value is not subtracted.

The concentration calculation units 242d calculate the concentration of a measurement target component using a plurality of sample correlation values obtained by the correlation value calculation units 242b.

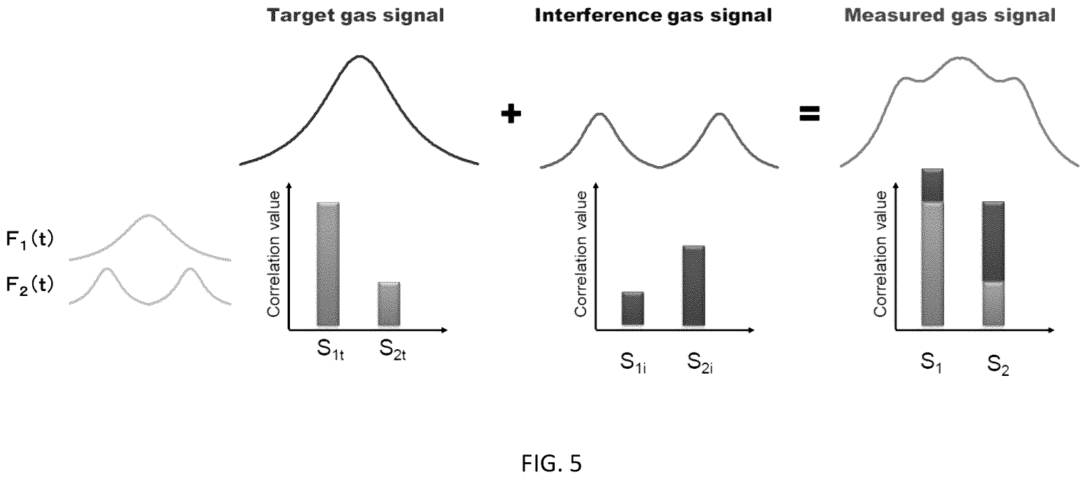

More specifically, the concentration calculation units 242d calculate the concentration of a measurement target component based on the plurality of sample correlation values obtained by the correlation value calculation units 242b, and on the plurality of individual correlation values stored in the storage units 242c. Even more specifically, the concentration calculation units 242d calculate the concentration of a measurement target component by solving simultaneous equations formed by the plurality of sample correlation values obtained by the correlation value calculation units 242b, the plurality of individual correlation values stored in the storage units 242c, and the respective concentrations of the measurement target component and each of the interference components. Note that FIG. 5 is a conceptual view showing a calculation of a concentration or partial pressure that is made by the concentration calculation units 242d using the individual correlation values and the sample correlation values.

In a case in which a single measurement target component (here, this is SiF₄) and a single interference component are contained in the measurement target gas, the concentration calculation units 242d solve the following simultaneous equations formed by the sample correlation values S₁′, S₂′ calculated by the correlation value calculation units 242b, the individual correlation values S₁ₜ, S₂ₜ, S₁ᵢ, S₂ᵢ in the storage units 242c, and the respective concentrations Cₜₐᵣ, Cᵢₙₜ of the measurement target component and the interference component. Note that s₁ₜ is an individual correlation value of the measurement target component in a first feature signal, s₂ₜ is an individual correlation value of the measurement target component in a second feature signal, s₁ᵢ is an individual correlation value of the interference component in the first feature signal, and s₂ᵢ is an individual correlation value of the interference component in the second feature signal.

$${s}_{1 t} {C}_{tar} + {s}_{1 i} {C}_{int} = {{S}_{1}}^{′} \qquad [ Equation 2 ]$$

$${s}_{2 t} {C}_{tar} + {s}_{2 i} {C}_{int} = {{S}_{2}}^{′}$$

Consequently, by performing the simple and reliable arithmetical operation of solving the simultaneous equations in the above Equation 2, it is possible to determine the concentration Cₜₐᵣ of the measurement target component (i.e., a reaction product) from which interference effects have been removed.

Note that, even in a case in which it can be assumed that two or more interference components are present, by adding the same number of individual correlation values as the number of interference components, and then solving the same original number of simultaneous equations as the number of types of component, it is still possible, in the same way, to determine the concentration of a measurement target component from which interference effects have been removed.

### [Structure of the State Evaluation Unit 3]

Next, the state evaluation unit 3 that evaluates the state of a Bosch process based on measurement values from the light absorption measurement units 2A and 2B will be described. Here, the measurement values from the light absorption measurement units 2A and 2B are concentrations of reaction products obtained by the signal processing units 242, or values relating to these concentrations.

The state evaluation unit 3 is equipped with an analog electrical circuit formed by amplifiers and the like, a CPU, digital electrical circuits formed by memory and the like, and AD converters and DA converters and the like that are interposed between these analog and digital electrical circuits. As a result of the CPU and peripheral devices thereof operating in mutual collaboration with each other in accordance with a predetermined state evaluation program that is stored in a predetermined area of the memory, the state evaluation unit 3 evaluates the state of a Bosch process. Moreover, which out of the deposition processing, anisotropic processing, or isotropic processing is the processing that is currently being performed in the chamber PC is transmitted to the state evaluation unit 3. Here, information about the processing currently being performed in the chamber PC is transmitted from a process control unit (not shown in the drawings) to a receiving unit 4, and this processing information received by the receiving unit 4 is then transmitted to the state evaluation unit 3. Furthermore, results of state evaluations (i.e., determinations) made by the state evaluation unit 3, or measurement values from the light absorption measurement units 2A and 2B can also be displayed on a display unit 5 such as display monitor or the like.

### (1) State Evaluation During a Deposition Step

The state evaluation unit 3 is able to evaluate the state of a Bosch process based on measurement values of SiF₄ obtained during the deposition step. More specifically, the state evaluation unit 3 is able to detect an end point of a Bosch process based on the size of a rise in the measurement value of SiF₄ obtained during the deposition step.

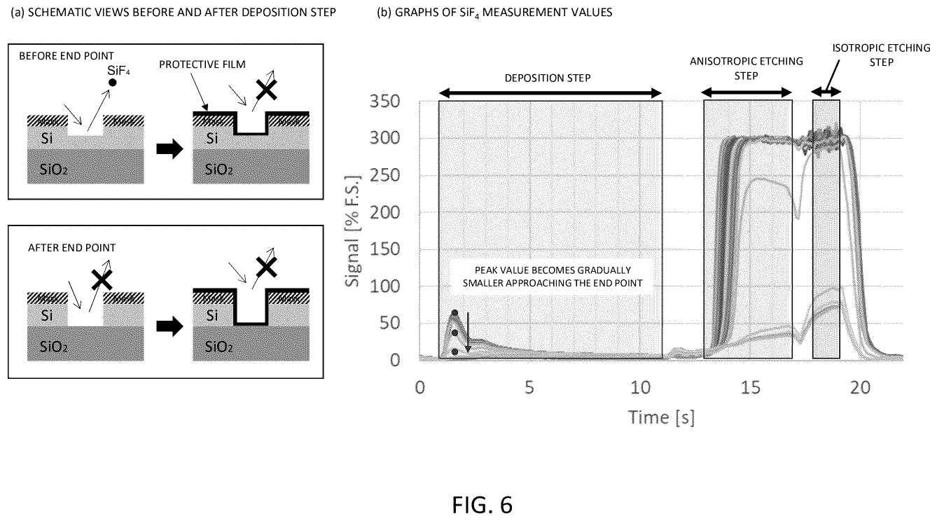

As is shown in FIG. 6 (a), before the end point of a Bosh process, SiF₄ is generated as a result of the F ions and/or F radicals that are generated from the C₄F₈ that is serving as the processing gas reacting with the exposed Si, and the generating of this SiF₄ becomes gradually less as the deposition of the protective film advances. In contrast, after the end point of the Bosch process, SiO₂ is exposed at the starting point of the deposition step so that it becomes more difficult for SiF₄ to be generated. In other words, as the end point of the Bosch process becomes progressively closer, the rise of the SiF₄ (i.e., the peak value) measured after the start of the deposition step becomes gradually smaller (see FIG. 6 (b)). Note that the rise of the SiF₄ refers not only to the peak value, but may also be a measurement value taken at a predetermined time after the deposition step has started. Note also that this predetermined time is from the start of the rise of the first peak of the SiF₄ measurement value after the start of the deposition step until the SiF₄ peak value has passed and the measurement value has stabilized. Using this characteristic, in a case in which the size of the rise in the SiF₄ measurement value (i.e., the peak value) during the deposition step drops below a first threshold value, the state evaluation unit 3 detects the end point of the Bosch process.

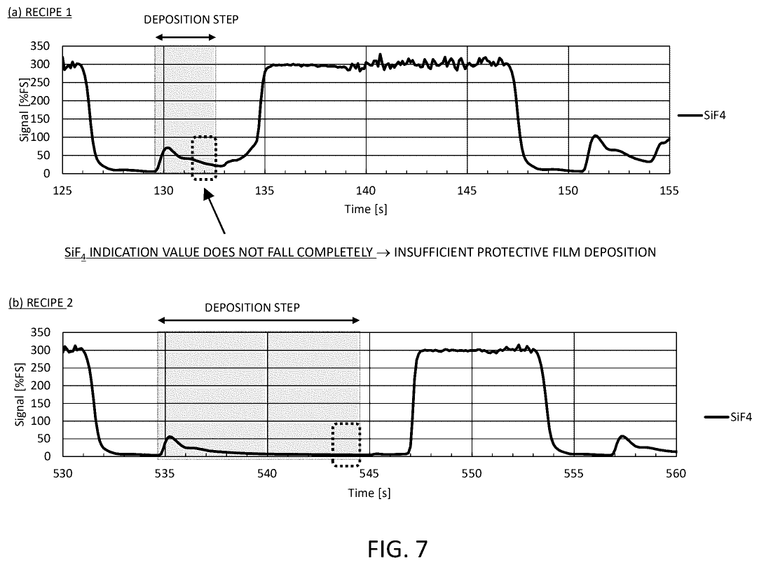

Moreover, as is described above, when the deposition step commences, the silicon (Si) is exposed so that SiF₄ is generated. As the deposition of the protective film advances, less and less SiF₄ is generated. Using this characteristic, in a case in which the SiF₄ measurement value during the deposition step falls below a second threshold value, the state evaluation unit 3 is able to determine that the deposition of the protective film is sufficient. Note that, in a case in which the deposition of the protective film is not sufficient (i.e., in a case in which the SiF₄ measurement value is greater than the second threshold value—see FIG. 7 (a)), then a profile abnormality such as an undercut (i.e., a profile in which the bottom portion of a groove or hole is bulging) or an inverse tapered profile or the like is generated. In this case, the state evaluation unit 3 is able to propose a recipe in which the deposition quantity in the deposition step is increased, such as by lengthening the deposition time during the deposition step or the like. Note that SiF₄ measurement values in a case in which the deposition time during the deposition step has been lengthened are shown in FIG. 7 (b).

### (2) State Evaluation of an Etching Step

The state evaluation unit 3 is able to evaluate the state of a Bosch process based on measurement values of SiF₄ obtained during the anisotropic etching step or the isotropic etching step. More specifically, the state evaluation unit 3 is able to detect that the object being etched has switched from the protective film to the silicon based on the measurement value of SiF₄ obtained during the anisotropic etching step.

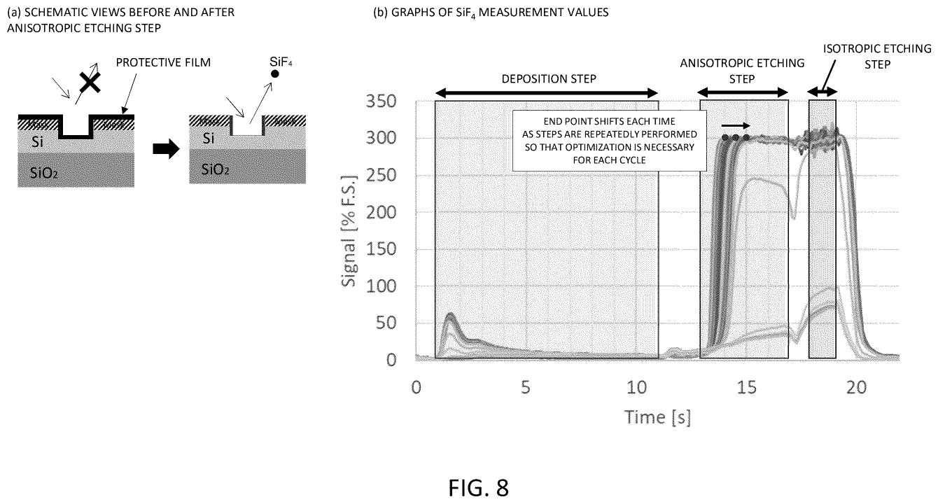

As is shown in FIG. 8 (a), because the protective film is etched at the time when the anisotropic etching step is started, SiF₄ is not generated. SiF₄ is generated once the etching of the protective film has ended. Using this characteristic, the state evaluation unit 3 is able to determine that the point in time when the SiF₄ measurement value during the anisotropic etching step reaches the maximum value is the end point of the protective film etching (see FIG. 8 (b)). Note that it is also possible for the state evaluation unit 3 to determine that the SiF₄ measurement value during the anisotropic etching step has reached a third threshold value (for example, a value in the vicinity of 150% F.S.), and by thereafter determining that a predetermined length of time has elapsed, to determine the end point of the protective film etching.

Moreover, the state evaluation unit 3 is also able to detect the end point of a Bosch process based on measurement values of SiF₄ obtained during the anisotropic etching step or the isotropic etching step. For example, in a case in which a measurement value of SiF₄ obtained during the anisotropic etching step or the isotropic etching step falls below a fourth threshold value, the state evaluation unit 3 is able to detect the end point of a Bosch process. In this case, the state evaluation unit 3 is able to detect the end point of a Bosch process during the above-described deposition step, and to thereby perform a double end point detection so as to stop a Bosch process at a more optimal end point.

Furthermore, in a case in which a measurement value of CO during the isotropic etching step falls below a threshold value, the state evaluation unit 3 is able to determine that the protective film is not sufficient. Note that the CO during the isotropic etching step is derived from carbon (C) contained in the protective film.

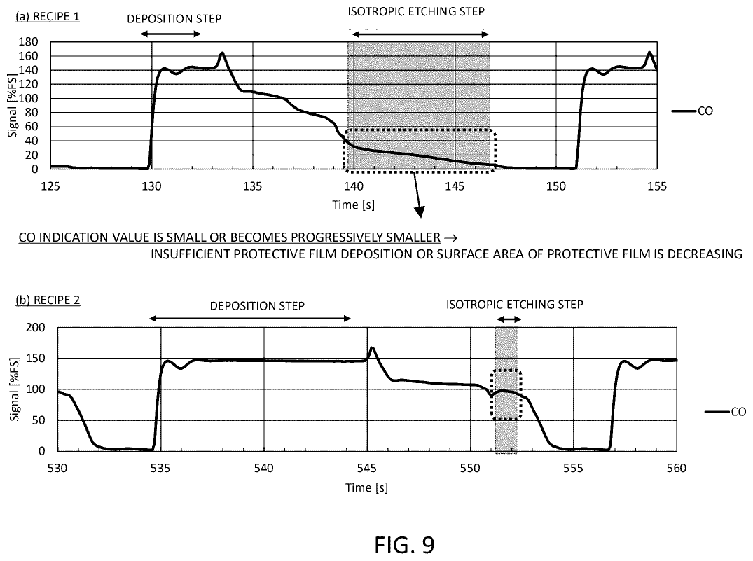

For example, in a case in which the CO measurement value is below a fifth threshold value (see FIG. 9 (a)), the state evaluation unit 3 is able to determine that the deposition of the protective film is not sufficient. Note that, in a case in which the deposition of the protective film is not sufficient, then portions where the protective film is not present are etched and a profile abnormality such as an inverse tapered profile or the like is generated. Moreover, in a case in which the CO measurement value gradually decreases (see FIG. 9 (a)), the state evaluation unit 3 is able to determine that the surface area of the protective film is decreasing. Note that, if the surface area of the protective film decreases, the silicon in the portions where the protective film has been peeled away is etched and a profile abnormality such as an undercut (i.e., a profile in which the bottom portion of a groove or hole is bulging) or the like is generated. In these cases, the state evaluation unit 3 is able to propose a recipe in which the deposition quantity in the deposition step is increased, such as by lengthening the deposition time during the deposition step or the like, and/or a recipe in which the extent of the etching of the protective film is decreased, such as by shortening the etching time of the protective film (i.e., the length of time of the isotropic etching step). Note that CO measurement values in a case in which the deposition step has been extended and the etching time during the isotropic etching step has been shortened are shown in FIG. 9 (b).

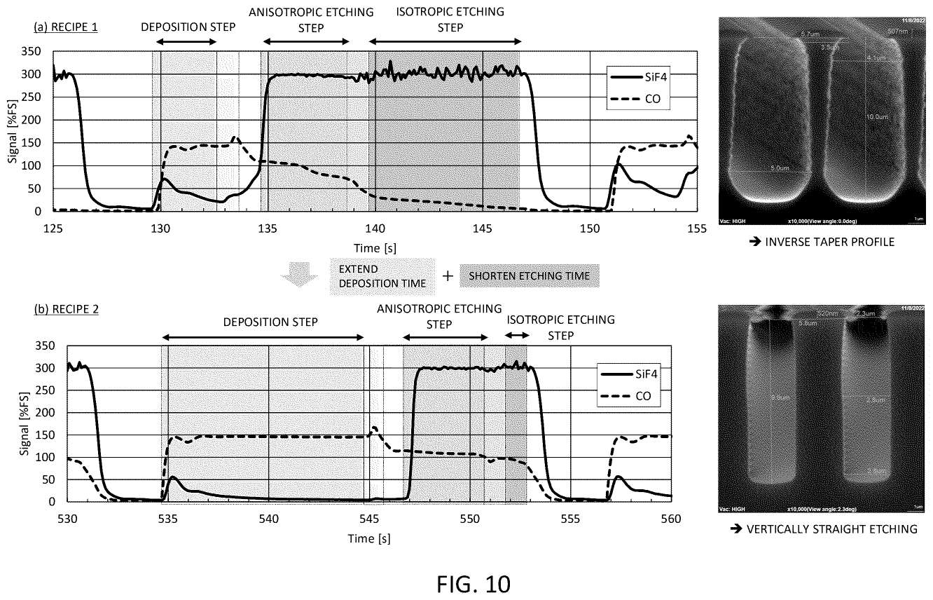

As is shown in FIG. 10 (a), in a case in which the state evaluation unit 3 has determined that the SiF₄ measurement value during the deposition step is greater than a threshold value and the deposition of the protective film is insufficient, and also that the CO measurement value during the isotropic etching step is less than a threshold value and the protective film is insufficient, then the groove formed by this etching has an inverse tapered profile.

In contrast to this, the results obtained when the recipes of the deposition step and the isotropic etching step are optimized based on the above-described determinations by the state evaluation unit 3 are shown in FIG. 10 (b). Here, the deposition time in the deposition step has been lengthened, and the etching time in the isotropic etching step has been shortened. By optimizing the recipe in this way, the grooves formed by the etching are etched vertically straight, so that the inverse tapered profile can be improved.

### Effects Obtained from the Present Embodiment

According to the process evaluation apparatus 100 of the present embodiment that has the above-described structure, because laser light is irradiated onto a gas that contains a reaction product generated during a Bosch process and this reaction product is then measured, and then, based on the measurement value of this reaction product, the state of the Bosch process is evaluated, it is possible to appropriately adjust the fabrication time and/or fabrication conditions in at least one of a deposition step, an isotropic etching step, or an anisotropic etching step in a Bosch process. As a result, it is possible to make the fabricated profile of a groove or hole being formed in a substrate in a Bosch process a desired profile, and to thereby improve the performance of a device that utilizes the substrate thus fabricated.

### Additional Embodiments

For example, in the above-described embodiment a structure is employed in which both SiF₄ and CO are measured as reaction products using the laser absorption measurement units 2A and 2B, however, it is also possible to employ a structure in which only one of SiF₄ or CO is measured as a reaction product. In addition, should another reaction product be generated, then it is also possible for that reaction product to be measured.

Furthermore, the laser absorption measurement units 2A and 2B of the above-described embodiment measure a concentration of a reaction product or a value related to this concentration, however, it is also possible for a partial pressure of a reaction product in a measurement target gas or a value related to this partial pressure to be measured. In this case, output values from the laser absorption measurement units 2A and 2B are the partial pressure of the reaction product obtained by the signal processing unit 242 or values relating to this partial pressure.

Additionally, it is also possible to employ a structure in which a Bosch process is controlled in real time based on results from state evaluations (i.e., determinations) made by the state evaluation unit 3. For example, if it is determined by the state evaluation unit 3 that the deposition of the protective film during the deposition step is sufficient, then it is possible for the deposition step to be ended and for the process to transit to the subsequent anisotropic etching step.

Furthermore, in the above-described embodiment a structure is employed in which the measurement units 2 are incorporated into the exhaust pipe H of the process chamber PC, however, it is also possible to employ a structure in which the measurement units 2 are provided on a bypass pipe that branches off from the exhaust pipe H, or a structure in which they are provided on a dedicated measurement pipe that is connected to the process chamber PC separately from the exhaust pipe H. Moreover, it is also possible to employ a structure in which a pair of multiple reflection mirrors M1 and M2 are provided inside the process chamber PC, or for these to be connected to the surrounding walls of the process chamber PC such as to the side walls or upper wall or the like thereof.

In addition, it is also possible for a single computer (i.e., an information processing device) to be provided with the functions of the signal processing devices 24 of the laser absorption measurement units 2A and 2B, and the state evaluation unit 3 of the above-described embodiment.

The light absorption measurement units of the above-described embodiment irradiate laser light, however, it is also possible for them to irradiate a different light than laser light.

Furthermore, it should be understood that the present invention is not limited to the above-described embodiments, and that various modifications and the like may be made thereto insofar as they do not depart from the spirit or scope of the present invention.

### REFERENCE CHARACTERS LIST

100 Process Evaluation Apparatus
2A, 2B Laser Absorption Measurement Units
3 State Evaluation Unit
PC Chamber
H Exhaust Pipe

What is claimed is:

1. A process evaluation apparatus that evaluates a process in which are repeatedly performed a deposition step in which a protective film is deposited on a substrate, an anisotropic etching step in which a portion of the protective film is removed by means of anisotropic etching, and an isotropic etching step in which the substrate that has been exposed due to the protective film being removed is removed by means of isotropic etching, comprising:
light absorption measurement units that irradiate light onto a gas containing a reaction product that has been generated during the process, and then measure the reaction product based on an absorption of this light; and
a state evaluation unit that, based on measurement values obtained by the light absorption measurement unit, evaluates a state of the process.

2. The process evaluation apparatus according to claim 1, wherein
the process is employed in order to etch silicon, and
the light absorption measurement units measure a first reaction product generated as a result of the silicon being etched, and
the state evaluation unit evaluates the state of the process based on measurement values of the first reaction product obtained by the light absorption measurement units.

3. The process evaluation apparatus according to claim 2, wherein
the state evaluation unit evaluates the state of the process based on measurement values of the first reaction product obtained during the deposition step.

4. The process evaluation apparatus according to claim 2, wherein
the state evaluation unit detects an end point of the process based on a size of a rise in the measurement values of the first reaction product obtained during the deposition step.

5. The process evaluation apparatus according to claim 2, wherein,
in a case in which the measurement values of the first reaction product obtained during the deposition step are equal to or less than a threshold value, the state evaluation unit determines that a deposition of the protective film is sufficient.

6. The process evaluation apparatus according to claim 2, wherein the state evaluation unit evaluates the state of the process based on measurement values of the first reaction product obtained during the anisotropic etching step or the isotropic etching step.

7. The process evaluation apparatus according to claim 6, wherein
the state evaluation unit detects that an object being etched has switched from the protective film to silicon based on measurement values of the first reaction product obtained during the anisotropic etching step.

8. The process evaluation apparatus according to claim 6, wherein
the state evaluation unit detects an end point of the process based on measurement values of the first reaction product obtained during the anisotropic etching step or the isotropic etching step.

9. The process evaluation apparatus according to claim 2, wherein the state evaluation unit measures SiF₄ as the first reaction product generated as a result of silicon being etched.

10. The process evaluation apparatus according to claim 1, wherein the light absorption measurement units measure a second reaction product that is generated as a result of the protective film being etched, and,
in a case in which measurement values of the second reaction product obtained by the light absorption measurement units during the isotropic etching step are equal to or less than a threshold value, the state evaluation unit determines that the protective film is insufficient.

11. The process evaluation apparatus according to claim 10, wherein
the protective film is formed by a compound that contains carbon, and
the light absorption measurement units measure any one of CO, CO₂, or CF₄ as the second reaction product that is generated as a result of the protective film being etched.

12. The process evaluation apparatus according to claim 1, wherein the light absorption measurement units are provided in an exhaust pipe that is connected to a chamber where the process is performed.

13. The process evaluation apparatus according to claim 1, wherein the light absorption measurement units irradiate laser light onto the gas, and measure the reaction product based on an absorption of that laser light.

14. A process evaluation method in which is evaluated a process in which are repeatedly performed a deposition step in which a protective film is deposited on a substrate, an anisotropic etching step in which a portion of the protective film is removed by means of anisotropic etching, and an isotropic etching step in which the substrate that has been exposed due to the protective film being removed is removed by means of isotropic etching, wherein light is irradiated onto a gas containing a reaction product that has been generated during the process, and the reaction product is then measured based on an absorption of this light, and
a state of the process is evaluated based on measurement values of the reaction product.

15. A computer-readable medium including a process evaluation program for evaluating a process in which are repeatedly performed a deposition step in which a protective film is deposited on a substrate, an anisotropic etching step in which a portion of the protective film is removed by means of anisotropic etching, and an isotropic etching step in which the substrate that has been exposed due to the protective film being removed is removed by means of isotropic etching, and that enables a computer to function as:
a state evaluation unit that evaluates a state of the process based on measurement values obtained by light absorption measurement units that irradiate light onto a gas containing a reaction product that has been generated during the process, and then measure the reaction product.
